(19) United States
(12) Patent Application Publication
(10) Pub. No.: US 2012/0022408 A1
(43) Pub. Date: Jan. 26, 2012

(54) Surgical Procedures Using Visual Images Overlaid with Visual Representations of Selected Three-Dimensional Data

(75) Inventors: Jean-Pierre Hubschman, Beverly Hills, CA (US); Steven Schwartz, Los Angeles, CA (US); Jason T. Wilson, Los Angeles, CA (US); Tsu-Chin Tsao, Manhattan Beach, CA (US); James S. Gibson, Manhattan Beach, CA (US)

(73) Assignee: VANTAGE SURGICAL SYSTEMS, INC.

(21) Appl. No.: 13/169,072

(22) Filed: Jun. 27, 2011

Related U.S. Application Data
(63) Continuation-in-part of application No. 13/164,671, filed on Jun. 20, 2011.
(60) Provisional application No. 61/358,780, filed on Jun. 25, 2010.
(60) Provisional application No. 61/356,150, filed on Jun. 18, 2010.

(51) Int. Cl. A61F 9/013; A61B 18/18; A61B 17/00; A61F 9/008; A61B 5/107

(52) U.S. Cl. 600/587; 606/107; 606/166; 606/5; 606/1; 606/10

(57) ABSTRACT

Embodiments of the invention are directed to improved surgical procedures such as ophthalmic procedures that utilize overlaid visual representations of three-dimensional data or other data with direct or indirect visual images of a surgical region (e.g. the eye) to provide improved information to a surgeon to speed surgical procedures and/or to provide improved outcomes of those procedures. In some embodiments, ophthalmic procedures are combined cataract removal and astigmatism reduction procedures. In other embodiments the ophthalmic procedures are corneal refractive surgical procedures that reshape the cornea to reduce astigmatism or other aberrations. In some ophthalmic procedures the three-dimensional data is topography data associated with the anterior surface of the cornea. In some embodiments, the three-dimensional data may be enhanced or replaced with aberrometric data associated with the optical path of the eye.

### RELATED APPLICATIONS

This application claims the benefit of U.S. Provisional Patent Application No. 61/358,780, filed Jun. 25, 2010 and is a continuation-in-part of U.S. patent application Ser. No. 13/164,671 filed Jun. 20, 2011 which in turn claims benefit of U.S. Provisional Patent Application No. 61/356,150 filed Jun. 18, 2010. These referenced applications are incorporated herein by reference as if set forth in full herein.

### FIELD OF THE INVENTION

The present invention relates generally to the field of surgical procedures and more particularly to surgical procedures involving the provision of enhanced visual information to surgeons in the form of overlaid (i.e. composite) images formed from the overlaying of at least first and second image components wherein the first image component is visual image of at least a portion of a surgical working area while the second component is an electronically displayed image that includes information not visually found within in the at least portion of the surgical working area. Particular embodiments of the invention are directed to ophthalmic surgical procedures and systems and more particularly to surgical procedures where corneal topographic data or other non-visual data is processed into a visual form and is combined with visual data to produce enhanced visual images allowing improved surgical procedures and outcomes. Some particular embodiments are focused on improved procedures for placing intra-ocular lenses (IOLs) including toric IOLs and/or enhanced methods of understanding refractive characteristics of the cornea allowing operative guidance of any corneal refractive surgery or other type of refractive surgery.

### BACKGROUND OF THE INVENTION

Surgical procedures: (1) involve certain risks to the patient, (2) take a certain time to perform, (3) take a certain experience or skill level by a surgeon, (4) result in the collateral damage of healthy tissue, (5) result in the excess removal of healthy tissue, (6) result in the inadequate removal of unhealthy tissue, (7) result in the failure to fulfill the surgical goal, (8) require prolonged recovery times, (9) result in extended periods of disability, and/or (10) result in the need for extended therapy. If a surgeon could be provided with more information during the performance of a procedure, be provided with that information in a more timely manner, and/or be provided with that information in a more accessible manner, many such procedures could: (1) be performed with less risk to the patient, (2) be performed more quickly, (3) be performed by a surgeon with less experience or skill, (4) result in reduced collateral damage, (5) result in removal of less healthy tissue, (6) result in more complete removal of unhealthy tissue, (7) result in higher probability of fulfilling the surgical goal, (8) result in less recovery time, (9) result in less disability or shortened periods of disability, and/or (10) result in less need for physical therapy. A need exists in the surgical arts for a method of providing more information, providing this additional information in a timely manner, and/or providing this information in a more accessible manner.

In cataract patients with astigmatism, it is critical for intraocular lenses (IOLs) to be placed precisely along a definite axis to correct the corneal distortion, to maximize the benefit of the surgery and to minimize the risk of the patient needing glasses after the operation. Such distortions can be measured very accurately using commonly available topographic measurement tools. The IOL placement techniques commonly used by surgeons presume that the position of the eye and the cornea remain constant between the time the topographic measurement is recorded and the operation time when the patient is lying down. In reality, the eye may rotate as much as 10-20 degrees which could lead to non-optimal placement of IOLs. A need exists for improved methods for placement of IOLs.

In patients undergoing corneal refractive surgery such as LASIK or RK, the accuracy of the location, depth and size of the cuts (through incisions or laser reshaping) are critical for favorable outcomes. The surgeon's decisions on these rely heavily on previously recorded topography information which may change during the surgery. Such reliance may lead to unfavorable outcomes due to movement of the eye and/or due to changes to the shape of the cornea between recording of topographic data and successive cutting steps. A need exists for improved methods for performing such surgeries such that likelihood of most favorable outcomes are enhanced.

### SUMMARY OF THE INVENTION

It is an object of some embodiments of the invention to provide an improved surgical procedure wherein the provision of more information to the surgeon, the more timely provision of the information, and/or the more accessible provision of the information results in the procedure being performed with less risk to the patient.

It is an object of some embodiments of the invention to provide an improved surgical procedure wherein the provision of more information to the surgeon, the more timely provision of the information, and/or the more accessible provision of the information results in the procedure being performed more quickly

It is an object of some embodiments of the invention to provide an improved surgical procedure wherein the provision of more information to the surgeon, the more timely provision of the information, and/or the more accessible provision of the information results in the procedure being successfully performable by a surgeon with less experience or skill,

It is an object of some embodiments of the invention to provide an improved surgical procedure wherein the provision of more information to the surgeon, the more timely provision of the information, and/or the more accessible provision of the information results in reduced collateral damage,

It is an object of some embodiments of the invention to provide an improved surgical procedure wherein the provision of more information to the surgeon, the more timely provision of the information, and/or the more accessible provision of the information results in the removal of less healthy tissue,

It is an object of some embodiments of the invention to provide an improved surgical procedure wherein the provision of more information to the surgeon, the more timely provision of the information, and/or the more accessible provision of the information results the more complete removal of unhealthy tissue,

It is an object of some embodiments of the invention to provide an improved surgical procedure wherein the provision of more information to the surgeon, the more timely provision of the information, and/or the more accessible provision of the information results in higher probability of fulfilling the surgical goal,

It is an object of some embodiments of the invention to provide an improved surgical procedure wherein the provision of more information to the surgeon, the more timely provision of the information, and/or the more accessible provision of the information results in less recovery time,

It is an object of some embodiments of the invention to provide an improved surgical procedure wherein the provision of more information to the surgeon, the more timely provision of the information, and/or the more accessible provision of the information results in less disability or shortened periods of disability,

It is an object of some embodiments of the invention to provide an improved surgical procedure wherein the provision of more information to the surgeon, the more timely provision of the information, and/or the more accessible provision of the information results in less need for physical therapy.

It is an object of some embodiments of the invention to provide improved methods for placing intraocular lenses (IOLs) during cataract surgery or other vision improvement surgeries.

It is an object of some embodiments of the invention to provide improved methods of placing IOLs that may be sensitive to orientation misalignments.

It is an object of some embodiments of the invention to provide improved methods of orienting toric IOLs.

It is an object of some embodiments of the invention to provide an improved method of performing corneal refractive surgery.

It is an object of some embodiments of the invention to provide improved visual feedback on the results of surgical steps to aid a surgeon in determining the need for, and in taking, further surgical steps.

It is an object of some embodiments of the invention to provide improved outcomes for patients undergoing surgeries that include the placement of one or more toric IOLs (e.g. to correct for astigmatism) where they may be used as replacements for the eye's natural lens, be used as replacements for a previously placed IOL, or be used placed and removed IOL, be used to supplement refraction by being located in the placement in the anterior chamber (behind the cornea and in front of the iris) or in the posterior chamber (behind the iris and in front of the crystalline lens) of the eye.

Other objects and advantages of various embodiments of the invention will be apparent to those of skill in the art upon review of the teachings herein. The various embodiments and aspects of the invention, set forth explicitly herein or otherwise ascertained from the teachings herein, may address one or more of the above objects alone or in combination, or alternatively may address some other object ascertained from the teachings herein. It is not intended that all objects be addressed by any single embodiment or aspect of the invention even though that may be the case with regard to some embodiments and aspects.

A first aspect of the invention provides an ophthalmic procedure involving placement of an intraocular lens within an eye of a patient, including: (a) obtaining at least one instance of topographic data of the anterior surface of the cornea of the eye of the patient; (b) processing the topographic data to obtain at least one instance of a desired computer generated visual representation; (c) overlaying visual images of a selected portion of the eye and selected features of the intraocular lens with the at least one instance of the computer generated visual representation to produce overlaid visual images, wherein the overlaying comprises use of markerless tracking; (d) inserting at least one intraocular lens into the eye of the patient at a desired location, wherein the at least one intra ocular lens comprises at least one toric intraocular lens; and (e) rotating the intraocular lens to a desired orientation relative to the eye using the overlaid visual images.

Numerous variation of the first aspect of the invention are possible and include for example: (1) the overlaying occurring a plurality of times and the using the overlaid images uses a plurality of updated images; (2) visual images being real time images, the at least one instance of obtaining and the at least one instance of processing being single instances, the overlaying occurring a plurality of times, and the using the overlaid images using a plurality of updated images; (3) the visual images being real time images, the at least one instance of obtaining and processing each comprising a plurality of instances, the overlaying occurs a plurality of times, and the using the overlaid images using a plurality of updated images; (4) the overlaying including positioning and orienting to provide an alignment of optical axes within a desired tolerance; (5) the at least one intraocular lens comprising a plurality of lenses and at least two of the plurality of intraocular lenses being toric lenses; (6) the procedure of claim 1 wherein the at least one intraocular lens comprises a plurality of lenses and the plurality of intraocular lenses comprise at least one lens that is substantially insensitive to rotational orientation; (7) the inserting locates at least one of the at least one intraocular lens within the capsule of the lens of the eye; (8) the inserting locates at least one of the at least one intraocular lens within the posterior chamber of the eye; (9) the inserting locates at least one of the at least one intraocular lens within an anterior chamber of the eye; (10) the procedure comprising a cataract surgery; (11) the procedure comprising a cataract surgery with astigmatism correction; (11) the procedure comprising reshaping of the anterior surface of the cornea; (13) use of aberrometric data; (14) the visual images of the selected portion of the eye and of the selected portion of the intraocular lens being direct visual images and (i) wherein the direct visual images are optionally viewed by a surgeon through an eye piece on a microscope and the overlaying occurs optically, or (ii) wherein the direct visual images are optionally viewed by a surgeon on a screen and the overlaying occurs optically, or (iii) wherein the overlaying that occurs optically via the merging of two separate optical paths optionally uses a beam splitter; (15) the visual images being first captured electronically by a camera and then electronically reproduced into visual images that are displayed for using and wherein prior to being electronically reproduced the captured visual images are optionally overlaid with the computer generated visual representation; and (16) the processing the topographic data including use of markerless tracking methods.

A second aspect of the invention provides an ophthalmic procedure involving reshaping the anterior surface of a cornea of an eye of a patient, comprising: (a) obtaining topographic data corresponding to a topology of the anterior surface of the cornea of the eye of the patient; (b) processing the topographic data into a desired computer generated visual representation; (c) overlaying a current visual image of a selected portion of the eye and a current computer generated visual representation of the topographic data of the cornea of the eye of the patient to produce a current overlaid visual image for use in performing the procedure; (d) modifying the anterior surface of the cornea of the eye of the patient; and (e) repeating the steps of obtaining, processing, and overlaying one or more times and as necessary the step of modifying to bring a topographic configuration of the eye to a configuration that improves the optical performance of the eye.

Numerous variations of the second embodiment of the invention are possible and include, for example: (1) the modifying of the anterior surface occurring through at least one incision made in the anterior surface, and wherein the modifying of the anterior surface optionally occurs through a plurality of incisions wherein the topography of the anterior surface changes with each incision; (2) the modifying of the anterior surface occurring through laser ablation; (3) the visual images of the selected portion of the eye being direct visual images; (4) the visual images being direct visual images that are viewed by a surgeon through an eye piece on a microscope and the overlaying occurring optically and wherein the overlaying optionally occurs via the merging of two separate optical paths using at least one beam splitter; (5) the visual images being first captured electronically by a camera and then electronically reproduced into visual images that are displayed for use, and wherein prior to being electronically reproduced the captured visual images being overlaid with the computer generated visual representation; and (6) use of aberrometric data; and (7) the processing the topographic data including use of markerless tracking methods.

A third aspect of the invention provides an ophthalmic procedure to improve the refraction of light through the eye of a patient, including: (a) periodically obtaining three-dimensional data of at least one feature of the eye of the patient; (b) processing the periodic three-dimensional data into a current desired computer generated visual representation; (c) aligning a current visual image of at least a portion of the eye with the current computer generated visual representation using a markerless tracking algorithm; (d) overlaying the aligned current visual image and current computer generated representation to obtain an overlaid current image; (e) viewing the overlaid current image; and (f) modifying the physical configuration of a selected portion of the eye based at least in part on observations made during viewing of the overlaid current image; and (g) repeating steps (a)-(f) one or more times whereby the procedure results in improved refraction of light by the eye.

Numerous variations of the third aspect of the invention are possible and include, for example: (1) the modifying of the physical configuration of the selected portion of the eye including modifying the anterior surface of the cornea, and wherein the modifying optionally occurs through laser ablation; (2) the modifying occurs via at least one incision made in an anterior surface of a cornea of the eye, and wherein the at least one incision made in the anterior surface of the cornea optionally includes a plurality of incisions wherein the topography of the anterior surface changes with each incision; (3) the visual images of the selected portion of the eye being direct visual images, and wherein the direct visual images are viewed by a surgeon through an eye piece on a microscope and the overlaying occurs optically; (4) the visual images being images captured electronically by a camera and then electronically reproduced into visual images that are then subject to viewing, and wherein prior to being electronically reproduced, the captured visual images are optionally overlaid with the computer generated visual representation; (5) the modification comprising inserting a toric IOL into the eye; and (6) processing the periodic three-dimensional data includes use of markerless tracking methods.

A fourth aspect of the invention provides an ophthalmic procedure to improve the refraction of light through the eye of a patient, including: (a) periodically obtaining aberrometer data for at least a portion of an optical path of the eye; (b) processing the periodic aberrometer data into a current desired computer generated visual representation; (c) aligning a current visual image of at least a portion of the eye with the current computer generated visual representation using markerless tracking; (d) overlaying the aligned current visual image and current computer generated representation to produce a current overlaid image; (e) viewing the overlaid current images; (f) modifying the physical configuration of a selected portion of the eye based at least in part on observations made during viewing of the overlaid current image; and (g) repeating steps (a)-(f) one or more times whereby the procedure results in improved refraction of light by the eye.

Numerous variations of the fourth embodiment of the invention exist and include, for example: (1) the modifying of the physical configuration of the selected portion of the eye including modifying an anterior surface of a cornea of the eye; (2) the modifying an anterior surface of a cornea of the eye occurs via at least one incision made in the anterior surface of the cornea; (3) the modifying an anterior surface of a cornea of the eye and occurs via a plurality of incisions wherein a topography of the anterior surface changes with each incision; (4) the modifying an anterior surface of a cornea of the eye and occurs through laser ablation; (5) the visual images of the selected portion of the eye being direct visual images, and wherein the direct visual images are optionally viewed by a surgeon through an eye piece on a microscope and the overlaying optionally occurs optically; (6) the visual images being images captured electronically by a camera and then electronically reproduced into visual images that are then subject to viewing, and wherein prior to being electronically reproduced the captured visual images are optionally overlaid with the aberrometer data; (7) the modification comprising inserting a toric IOL into the eye; and (8) the periodically obtaining aberrometer data including use of markerless tracking methods.

A fifth aspect of the invention provides a surgical procedure, including: (a) acquiring three-dimensional data related to a surgical region of a body of a patient; (b) processing the three-dimensional data into a desired computer generated visual representation; (c) providing a visual image of the surgical region; (d) overlaying the visual image of the surgical region with the computer generated visual representation of the topographic data to obtain an overlaid visual image for use in performing the procedure; (e) executing a diagnostic, preventative, or therapeutic action at the surgical region using a selected surgical instrument; and (f) repeating at least the steps of providing and overlaying one or more times in performing the surgical procedure, wherein the repeated providing and overlaying provides information about the surgical area that can be used by a surgeon in performing a next executing step.

Numerous variations of the fifth aspect of the invention exist and include, for example: (1) the repeating at least the steps of providing and overlaying including repeating the steps of acquiring, processing, providing, and overlaying, and wherein the acquiring, processing, providing, and overlaying provides information about the surgical area that can be used by a surgeon in performing a next executing step, and wherein the repeating of the steps optionally provides modified information based on an action taken in a previously performed executing step; (2) the overlaying comprises use of a markerless tracking algorithm; and (3) the executing step provides a step including a procedure selected from the group consisting of: (a) shaping of a selected tissue; (b) removing a selected portion of tissue; (c) analyzing a property of a selected portion of tissue; (d) dosing a selected portion of tissue with a selected material; (e) implanting a medical device, (f) irradiating a selected portion of tissue with visible light, (g) irradiating a selected portion of tissue with IR light, and (h) irradiating a selected portion of tissue with radiation from a radioactive source.

Other aspects of the invention will be understood by those of skill in the art upon review of the teachings herein. Other aspects of the invention may involve combinations of the above noted aspects of the invention. These other aspects of the invention may provide various combinations of the aspects presented above as well as provide other configurations, structures, functional relationships, and processes that have not been specifically set forth above. Other aspects may, for example, provide devices or systems for performing the above note procedural aspects.

### BRIEF DESCRIPTION OF THE DRAWINGS

FIG. 1 provides a schematic illustration of the steps of an example embodiment according to the first group of embodiments wherein visual images of the eye and selected portions of an IOL (as an example of a surgical area) are overlaid with corneal topography data (as an example of three-dimensional data) and the composite images (i.e. overlaid images) are used by a surgeon to aid in placing and/or orienting the IOL relative to the eye of the patient (e.g. use of enhanced data in the performance of an improved surgical procedure).

FIG. 2 provides a schematic illustration of the steps of an example embodiment according to the third group of embodiments wherein visual images of at least a portion of the eye of a patient (as an example of a surgical area) are overlaid with corneal topographical data (as an example of three-dimensional data) is updated periodically during the performance of a corneal shaping procedure (e.g. as an example surgical procedure) to provide a plurality of updated composite images to a surgeon to aid in the shaping of the cornea).

### DETAILED DESCRIPTION OF PREFERRED EMBODIMENTS

### Methods for Highly Accurate Placement of Orientation Sensitive IOLs

A first specific group of embodiments of the invention is directed to the use of previously recorded three-dimensional data (e.g. topographic data) for the anterior surface of the cornea (along with its spatial relationship to the rest of the eye) in combination with real-time image data of the eye. The three-dimensional data is processed by a programmed computer to generate a visual representation of the three-dimensional data thereafter the visual representation is aligned with and overlaid on the image data from the eye so that the visual representation and image data may be viewed simultaneously. In some embodiments, the overlaying occurs based on electronic data associated with the visual representation of three-dimensional data and electronic data associated with the real-time image data of the eye. In other embodiments, the overlaying occurs optically based on two or more optical images presented along different optical paths that are merged (e.g. via one or more beam splitters and possibly different optical elements as well). In the most preferred embodiments of the invention, the positioning, aligning, and/or orienting of the visual representation and image data for the eye occurs via the use of markerless tracking algorithms though in other embodiments other methods may be used. Such markerless tracking algorithms are known in the art and have been described previously. See for example the Section entitled Markerless Tracking” in U.S. Pat. No. 7,428,318; U.S. Patent Pub. No. 2005-1190972; and Comport et al. IEEE Trans Visual. Compo Graph. 12(4); 615-628 (2006). Additional teachings concerning the overlaying of multiple images can be found in U.S. patent application Ser. No. 13/164,671 which is entitled “Augmented Reality Methods and Systems Including Optical Merging of a Plurality of Component Optical Images” and which was referenced in the related application section of this application. Each of these referenced applications, patents and publications is hereby incorporated herein by reference as if set forth in full herein.

In the methods of the first group of embodiments, an intraocular lens is inserted into a desired location within the eye of a patient, e.g. into the capsule after removal of the natural lens or removal of a previously placed IOL, in the anterior chamber, or into the posterior chamber. In cases where the intraocular lens is a toric lens (i.e. provides for astigmatic correction), the lens is rotated to a desired orientation relative to the orientation of the astigmatism of the eye to provide for reduced overall astigmatism along the optical path through the eye while using the overlaid visual representation and visual image for guidance. The overlaid representation and image data may be used to quickly and efficiently orient an axis of the IOL relative to an axis of the astigmatism associated with the surface of the cornea to provide an improved refractive light path for the eye (i.e. a light path with less overall astigmatism). In the most preferred embodiments elimination of distortion is the target while in other embodiments, minimization is the target, while in still other embodiments simply providing a reduction in astigmatism is the target.

In the first group of embodiments, the procedure may take on a variety of forms. It may, for example, be a combined cataract removal and astigmatism mitigation procedure involving phacoemulisification, capsulorrhexis, and IOL placement. It may be procedure to reduce astigmatism in a patient that has previously undergone cataract surgery where a toric lens will replace a previously placed toric or non-toric lens, where a toric lens will be placed within an anterior chamber, within a posterior chamber, or within the capsule of the eye wherein a crystalline lens has been removed, or within two or more of these locations (e.g. one IOL implanted with the anterior chamber and one within the posterior chamber) In still other procedures, toric lenses and/or other lenses (e.g. multifocal IOLs and/or adaptive IOLs) may used to provide reduced astigmatism, improved accommodation, or other refractive path improvements without being part of a cataract removal procedure.

In variations of the first group of embodiments, the three-dimensional data may be obtained by corneal topography, photokeratoscopy, or videokeratography methods known to those of skill in the art. In still other variations optical coherence tomography (OCT) methods may be used. In still other variations other obtainment methods may be used such as, for example, confocal microscopy.

The obtained three-dimensional (e.g. topographic data) may be processed by techniques known to those of skill in the art to obtain visual representations of those features or attributes of those features.

The insertion of one or more intraocular lenses into the eye of the patient can be performed using procedures known to those of skill in the art, e.g. capsulorrhexis, phacoemulsification, implantation of the IOL, explantation of a previously placed IOL, and the like.

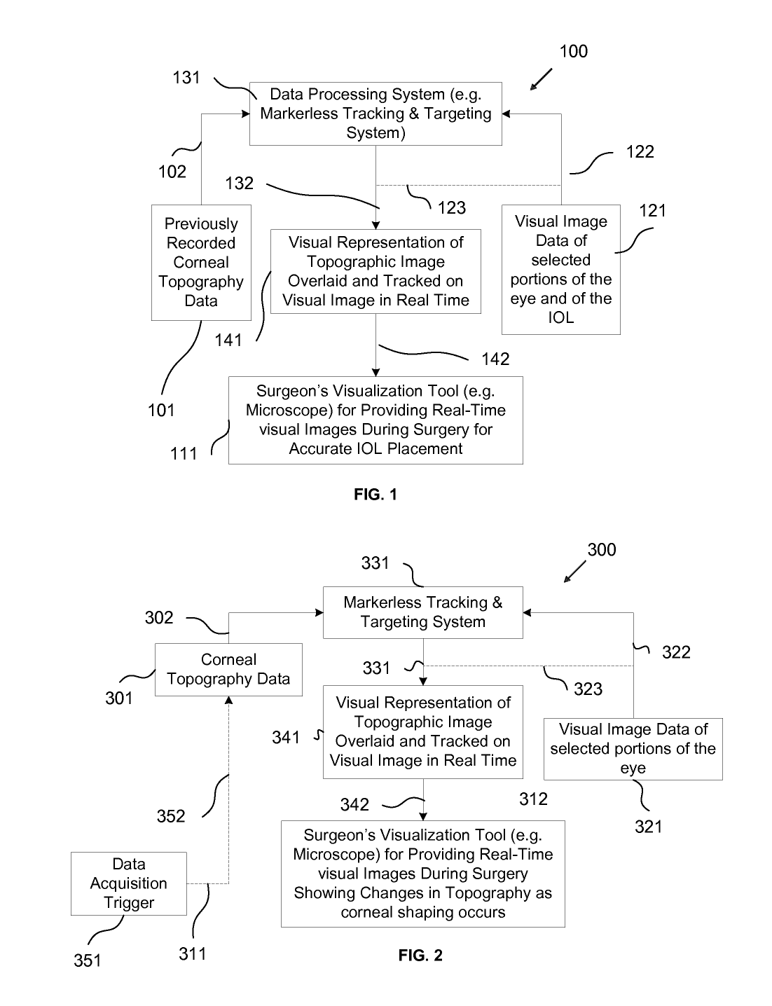

FIG. 1 provides a schematic block diagram of data, manipulations, and components used by a first group of embodiments of the invention. The devices include a data processing device or system 131, an electronic optical display device (part of block 141), a surgeon's visualization tool 111 for viewing composite images, a source (e.g. part of block 121) of visual image data associated with a surgical working area (i.e. the eye of a patient and IOL in this specific embodiment), and a source (e.g. part of block 101) of desired topographical data (i.e. corneal topography data in this specific embodiment).

The composite image to be viewed by the surgeon provides an image of the surgical area along with the enhanced information that the surgeon can use to provide one or more of improved surgical outcomes, shortened procedure time, reduced tissue damage, and the like. In the case of an IOL placement surgery, the composite image is formed from spatially overlaying of at least two component images to allow improved placement of IOLs and particularly improved placement of toric IOLs. The visualization tool 111 may provide for direct or indirect viewing of the eye or the patient and the IOL as it is being placed. In the case of direct visual images being supplied to the visualization tool 111, the visualization tool may include an image capture device for supplying data to block 121.

Procedure 100 also includes obtaining the second component data 101 i.e. corneal topography data in this specific embodiment). As noted in general above, this data may be obtained in a variety of ways such as by using, for example, corneal topography methods, photokeratoscopy methods, videokeratography methods, optical coherence tomography (OCT) methods, and/or confocal microscopy methods. Data 101 may be used as provided or it may undergo subsequent processing to put it in a desired form for further analysis, manipulation, and/or presentation. Data 101 is transmitted via path 102 to data processing system 131 which may be a programed computer or group of programmed computers, a digital signal processor or processors, or the like.

Procedure 100 also includes the provision of visual image data 121 of selected portions of the eye of the patient and of the IOL when it is in proximity to the eye). Two possible sources of image data for the eye exist. One source is an image capture device that may be associated with the surgeon's visualization tool 111 when the visualization tool directly views the surgical area. In such a case an optical path may be split from the path being observed by the surgeon so that it may be captured. Alternatively, the source maybe an image capture device that is independent of the surgeon's visualization tool 111 (e.g. when the visualization tool only provides for indirect viewing of the surgical area). As with data from block 101, data from block 121 is passed to data processing block 131 via line 122 and in some variations an optical image is passed through block 121 and fed into block 132. Block 131 represents the processing of visual image data for the surgical area and topographical data to yield data that can be used in providing the desired composite image. The result of the data processing may take on a number of different forms. In one form, topographical data has one or more of its position, alignment, size, and color manipulated for subsequent merging with the visual image data (in association with later block 131 processing) to produce composite image data or for subsequent reproduction as a visual image (in association with block 141) which is followed by optical merging (in association with block 141) to produce a composite optical image. In another form, both the topographical data and the visual image data are manipulated to achieve desired composite image data (e.g. shifting the visual image data to keep it centered and aligning the topographical data to the shifted visual image data). In either of these variations or numerous others, as noted previously, initial topographical data may be manipulated to produce alternative topographical data for overlaid presentation with the visual image data.

The processed data coming from block 131 is fed to block 141 where it is converted into a composite optical image. In some embodiment variations, block 141 may include an electronic optical display device that provides the composite optical image from composite image data and passes this composite optical image onto the surgeon's visualization tool for use by the surgeon. In other variations, block 141 may include an electronic optical display device for providing only an optical image of the desired topographical information to be displayed which may be combined with an optical image of the surgical area that is passed along (optional) path 123 to block 141. The optical image passed along path 123 may be a direct image of the surgical area that was optically split from the image that was used to produce the data that was fed from block 121 to block 131.

In some such variations, where optical merging is to occur, an image of the topography data, or an image created at least in part from the topography data, may be transformed into an optical image (i.e. a second component optical image) by an electronic optical image display device (not shown) and then optically merged to with an image of the of the surgical area as indicated in block 141. The image of the surgical area (i.e. a first component optical image) that is merged with the second component optical image may be either be (1) a direct image of the surgical area (i.e. an image that is directed solely along an optical path from the surgical area to the eye of an observer, in other words an optical image that does not undergo an intermediate electronic image capture and optical recreation before by an electronic image optical display device before reaching the eye of the observer), or (2) an indirect image of the surgical area (i.e. an optical image that moves along an optical path, is captured by an electronic image capture device, may or may not undergo manipulation, and is then reconverted into an optical image by an electronic image display device and from there continues along an optical path for viewing by an observer).

In other variations where electronic merging is to occur, merging occurs using data representing the first component image or images and data representing the at least one second component image with spatially merged data being sent to and displaced by an electronic image display device for viewing as a composite image by an observer (e.g. a surgeon).

In either variation, the data processing system 131 processes the first component data and the second component data to yield a desired registration or correlation of the data (e.g. positioning, alignment, and/or orienting) to allow subsequent merging of the data or images into composite images (i.e. enhanced images) for display to an observer (e.g. a surgeon). The enhanced data is used by the surgeon in performing the procedure to provide an improved surgical outcome.

In other embodiments, other methods of merging component images are possible some of which are described in the previously incorporated '671 application (e.g. methods for comparing optically merged images to ensure that the overlay calculations have provided adequate image registration. Devices and methods for extracting a portion of an optical image presented along an optical path are also described in the previously referenced '671 patent application.

In some variations of this first embodiment, some of the components and data manipulations set forth in FIG. 1 may be broken into multiple elements and manipulations while in other variations, the elements may be combined into single components and manipulations (e.g. system 131 and tool 111 may be a single element with necessary data manipulations and visual information.

In practice, the visual image data 121 are preferably presented in real time with rapid updates (e.g. once every few seconds, once every second, several times a second, and even several tens or even hundreds of times per second) while the topographic data may only be provided once per procedure and wherein the topographic data is continuously correlated to updated visual images so that composite images viewed by the surgeon maintain proper registration of components images (e.g. position, size, orientation, and the like).

In a second group of embodiments, the same procedures of the first group may be followed with the exception that the three-dimensional data or topographical data is recaptured periodically during the course of the surgery and the latest captured data is used in forming composite images so that the latest enhanced image presented to the surgeon shows updates that reflect any changes that have occurred to provide the surgeon with updated information that can be used in making improved procedural decisions and/or in taking improved procedural actions. In some variations, the three-dimensional data may be updated every several seconds or several times per second or as rapidly or nearly as rapidly as the visual image data so that composite images represent not only real-time visual images of the surgical image but substantially reali time images of the three-dimensional data as well.

In variations of the first and second embodiments, when performing procedures for improving the optical performance of the eye, aberration data along part or all of the optical path of the eye may also be obtained one or more times and a desired visual representation of attributes of the optical path overlaid with the images of the surgical working area to provide the surgeon with additional feedback on the effectiveness of the procedure.

In further variations of the first and second embodiments, updates of topographic or aberration data may be obtained at set time intervals or upon initiation by the surgeon.

Methods for Utilizing Corneal Topographic Data for Visual Feedback During Corneal Refractive Surgery

A third specific group of embodiments is directed to improved procedures for corneal refractive surgery. These embodiments like the first and second group of embodiments provide improved surgical outcomes by use of composite images to provide enhanced information, more accessible information, and/or more timely information to the surgeon to provide improved surgical outcomes wherein the composite images are formed from images of the surgical area overlaid with images derived from three-dimensional data that related to the intended surgical outcome. In these specific embodiments, the anterior surface of the cornea is reshaped. This reshaping may occur, for example, through incisions or by laser reshaping as is known in the art. In these embodiments topographic data for the anterior surface of the cornea is periodically obtained (e.g. via OCT or corneal topography). This topographic data is processed to yield a desired computer generated visual representation of the data. In performing the surgery, the surgeon directly or indirectly views a current visual image of a selected portion of the eye along with a current computer generated visual representation of the topographic data. The overlay (or composite image) of the visual image and visual representation are aligned, sized, and oriented, as with the first and second groups of embodiments. The overlaying preferably occurs using markerless tracking methods though use of other methods is possible in alternative embodiments.

With guidance from the overlay, the surgeon modifies the surface of the cornea by manual or computer controlled incision or ablation. Updating of the topographic data and visual representation provides the surgeon with real time, or near real time, updates as to the effects that the modifications are having on the shape of the cornea. As necessary, using successively updated overlaid images and visual representations, the surgeon continues making modifications until the overlaid images indicate that the modifications have resulted in the cornea taking on a shape that matches, or is within a desired tolerance of, a desired configuration.

FIG. 2 provides a schematic illustration of the steps of an example embodiment according to this third group of embodiments. As similarities exist between the first and third embodiments, elements of FIG. 2 which are similar to those of FIG. 1 are identified with similar reference numerals but wherein the numerals are updated to the 300 series. As noted above and in block 311, the procedure 300 provides for corneal shaping and shape monitoring using composite image information. As the corneal topography data in this embodiment is to be updated periodically FIG. 2 includes a data acquisition trigger 351 that is used to trigger re-acquisition of topography data

In variations of the third embodiment, aberration data may be obtained and overlaid to provide the surgeon with different or additional feedback on the effects of the modifications. In still other variations, IOLs may be placed at desired locations in the eye to provide additional enhancement to the refractive performance of the eye. Other variations noted above for the first and second embodiments may be employed in variations of the third embodiment, mutatis mutandis.

Additional Ophthalmic Surgical Procedures Involving Overlaid Visual Representations and Visual Image Data

A fourth group of embodiments is directed to ophthalmic procedures to provide improved refraction of light through the eye. In these embodiments, the eye undergoes physical modification to change the refractive or imaging properties of the eye (e.g. by adding in one or more IOLs or by reshaping the anterior surface of the cornea). In these methods, aberrometric data is obtained concerning at least a portion of the optical path through the eye and the data is processed to yield a desired computer generated visual representations of the data. In performing the procedure, the surgeon views the current visual image of a selected portion of the eye along with the current computer generated visual representation of the aberrometric data and uses that information to make surgical decisions and/or control physical modification activities. With each modification step, or after a number of modification steps, revised aberrometric data is obtained, processed and overlaid such that the optical results of the physical modification can be seen and one more next modification steps determined and/or executed. When the representation of the aberrometric data shows the imaging properties of the eye are within a desired target range, the procedure is completed.

Variations of the fourth embodiment are also possible and include, for example, elements that were included in the first-third embodiments and their variations, mutatis mutandis.

Generalized Surgical Procedures Using Overlaid Visual Representations of Three-Dimensional Data or Non-Visually Acquired Data with Surgical Field Image Data

Other embodiments may be directed to other medical procedures where selected tissue is to be shaped; removed; analyzed; dosed with a drug or other material, implanted with a medical device; irradiated with visible, IR, UV, or other radiation; or the like. These other procedure may for example include biopsy tissue extraction, tumor removal, infusion of therapeutic drugs or diagnostic materials, diagnostic procedures, infusion of drugs, optical tissue ablation, optically enhanced tumor destruction, or the like.

Such alternative embodiments involve the acquiring of three-dimensional data related to a surgical region of a body of a patient. This three-dimensional data may be obtained using an acquisition method that does not involve irradiation with visible light. The three-dimensional data is processed by a computer or other digital data processing device or system to create a desired computer generated visual representation of the data or of a portion of the data. A visual image is also provided for a surgical region of interest. The visual image may be accompanied by visual image data. The process of these alternative embodiments also involves overlaying the visual image of the surgical region with the computer generated visual representation of the topographic data to obtain an overlaid visual image for use in performing the procedure. These alternative procedures also make use of the data in making decisions or in implementing such decisions, i.e. in executing surgical actions such as diagnostic, preventative, or therapeutic actions. These actions may be performed by a surgeon directly or robotically with or with surgeon control. The procedures of these alternative embodiments may also involve repeating some of the above steps to provide continued information updates to a surgeon. For example in some embodiments the visual data procurement and overlaying with three-dimensional data is repeated. In other embodiments, the visual data procurement, the three-dimensional data procurement, three-dimensional data processing, and overlaying is repeated to provide even a greater amount of enhanced data to the surgeon.

Further Comments and Conclusions

Though various portions of this specification have been provided with headers, it is not intended that the headers be used to limit the application of teachings found in one portion of the specification from applying to other portions of the specification. For example, it should be understood that alternatives acknowledged in association with one embodiment, are intended to apply to all embodiments to the extent that the features of the different embodiments make such application functional and do not otherwise contradict or remove all benefits of the adopted embodiment. Various other embodiments of the present invention exist. Some of these embodiments may be based on a combination of the teachings herein with various teachings incorporated herein by reference.

The methods described herein may be used in combination with the methods set forth in U.S. patent application Ser. No. 13/169,076, by Jean P. HUBSCHMAN et al., filed concurrently herewith, having Docket No. VSSP-008US-A, and entitled “Surgical Procedures Using Instrument to Boundary Spacing Information Extracted from Real-Time Diagnostic Scan Data”. Further information about overlaying multiple visual images (whether they be from physical sources or from computer rendered images) can be found in the various patents, patent applications, and non-patent publications referenced herein (e.g. in the '671 application referenced herein above). These referenced patents, applications, and non-patent publications are each incorporated herein by reference as if set forth in full herein.

In view of the teachings herein, many further embodiments, alternatives in design and uses of the embodiments of the instant invention will be apparent to those of skill in the art. As such, it is not intended that the invention be limited to the particular illustrative embodiments, alternatives, and uses described above but instead that it be solely limited by the claims presented hereafter.

We claim:

1. An ophthalmic procedure involving placement of an intraocular lens within an eye of a patient, comprising:
(a) obtaining at least one instance of topographic data of the anterior surface of the cornea of the eye of the patient;
(b) processing the topographic data to obtain at least one instance of a desired computer generated visual representation;
(c) overlaying visual images of a selected portion of the eye and selected features of the intraocular lens with the at least one instance of the computer generated visual representation to produce overlaid visual images, wherein the overlaying comprises use of markerless tracking;
(d) inserting at least one intraocular lens into the eye of the patient at a desired location, wherein the at least one intra ocular lens comprises at least one toric intraocular lens; and
(e) rotating the intraocular lens to a desired orientation relative to the eye using the overlaid visual images.

2. The procedure of claim 1 wherein the overlaying occurs a plurality of times and the using the overlaid images uses a plurality of updated images.

3. The procedure of claim 1 wherein the visual images are real time images, the at least one instance of obtaining and the at least one instance of processing are single instances, the overlaying occurs a plurality of times, and the using the overlaid images uses a plurality of updated images.

4. The procedure of claim 1 wherein the visual images are real time images, the at least one instance of obtaining and processing each comprise a plurality of instances, the overlaying occurs a plurality of times, and the using the overlaid images uses a plurality of updated images.

5. The procedure of claim 1 wherein overlaying includes positioning and orienting to provide an alignment of optical axes within a desired tolerance.

6. The procedure of claim 1 wherein the at least one intraocular lens comprises a plurality of lenses and at least two of the plurality of intraocular lenses are toric lenses.

7. The procedure of claim 1 wherein the at least one intraocular lens comprises a plurality of lenses and the plurality of intraocular lenses comprise at least one lens that is substantially insensitive to rotational orientation.

8. The procedure of claim 1 wherein the inserting locates at least one of the at least one intraocular lens within the capsule of the lens of the eye.

9. The procedure of claim 1 wherein the inserting locates at least one of the at least one intraocular lens within the posterior chamber of the eye.

10. The procedure of claim 1 wherein the inserting locates at least one of the at least one intraocular lens within an anterior chamber of the eye.

11. The procedure of claim 1 wherein the procedure comprises a cataract surgery.

12. The procedure of claim 1 wherein the procedure comprises a cataract surgery with astigmatism correction.

13. The procedure of claim 1 wherein the procedure comprises reshaping of the anterior surface of the cornea.

14. The procedure of claim 1 additionally comprising the use of aberrometric data.

15. The procedure of claim 1 wherein the visual images of the selected portion of the eye and of the selected portion of the intraocular lens are direct visual images.

16. The procedure of claim 15 wherein the direct visual images are viewed by a surgeon through an eye piece on a microscope and the overlaying occurs optically.

17. The procedure of claim 15 wherein the direct visual images are viewed by a surgeon on a screen and the overlaying occurs optically.

18. The procedure of claim 15 wherein the overlaying that occurs optically via the merging of two separate optical paths using a beam splitter.

19. The procedure of claim 1 wherein the visual images are first captured electronically by a camera and then electronically reproduced into visual images that are displayed for using.

20. The procedure of claim 19 wherein prior to being electronically reproduced the captured visual images are overlaid with the computer generated visual representation

21. The procedure of claim 1 wherein the processing of the three-dimensional data comprises use of markerless tracking methods.

22. An ophthalmic procedure to improve the refraction of light through the eye of a patient, comprising:
(a) periodically obtaining three-dimensional data of at least one feature of the eye of the patient;
(b) processing the periodic three-dimensional data into a current desired computer generated visual representation;
(c) aligning a current visual image of at least a portion of the eye with the current computer generated visual representation using a markerless tracking algorithm;
(d) overlaying the aligned current visual image and current computer generated representation to obtain an overlaid current image;
(e) viewing the overlaid current image;
(f) modifying the physical configuration of a selected portion of the eye based at least in part on observations made during viewing of the overlaid current image; and
(g) repeating steps (a)-(f) one or more times whereby the procedure results in improved refraction of light by the eye.

23. The procedure of claim 22 wherein the modifying of the physical configuration of the selected portion of the eye comprises modifying the anterior surface of the cornea.

24. The procedure of claim 22 wherein the modifying occurs via at least one incision made in an anterior surface of a cornea of the eye.

25. The procedure of claim 24 wherein the at least one incision made in the anterior surface of the cornea comprises a plurality of incisions wherein the topography of the anterior surface changes with each incision.

26. The procedure of claim 23 wherein the modifying occurs through laser ablation.

27. The procedure of claim 221 wherein the visual images of the selected portion of the eye are direct visual images.

28. The procedure of claim 27 wherein the direct visual images are viewed by a surgeon through an eye piece on a microscope and the overlaying occurs optically.

29. The procedure of claim 22 wherein the visual images are images captured electronically by a camera and then electronically reproduced into visual images that are then subject to viewing.

30. The procedure of claim 29 wherein prior to being electronically reproduced, the captured visual images are overlaid with the computer generated visual representation.

31. The procedure of claim 22 wherein the modification comprising inserting a toric IOL into the eye.

32. The procedure of claim 22 wherein the periodically obtaining three-dimensional data comprises periodically obtaining aberrometer data.

33. A surgical procedure, comprising:
(a) acquiring three-dimensional data related to a surgical region of a body of a patient;
(b) processing the three-dimensional data into a desired computer generated visual representation;
(c) providing a visual image of the surgical region;
(d) overlaying the visual image of the surgical region with the computer generated visual representation of the topographic data to obtain an overlaid visual image for use in performing the procedure;
(e) executing a diagnostic, preventative, or therapeutic action at the surgical region using a selected surgical instrument;
(f) repeating at least the steps of providing and overlaying one or more times in performing the surgical procedure, wherein the repeated providing and overlaying provides information about the surgical area that can be used by a surgeon in performing a next executing step.

34. The method of claim 33 wherein the repeating at least the steps of providing and overlaying comprises repeating the steps of acquiring, processing, providing, and overlaying, and wherein the acquiring, processing, providing, and overlaying provides information about the surgical area that can be used by a surgeon in performing a next executing step.

35. The method of claim 33 wherein the repeating of the steps provides modified information based on an action taken in a previously performed executing step.

36. The method of claim 33 wherein the overlaying comprises use of a markerless tracking algorithm.

37. The method of claim 33 wherein the executing step provides a step comprising a procedure selected from the group consisting of: (1) shaping of a selected tissue; (2) removing a selected portion of tissue; (3) analyzing a property of a selected portion of tissue; (4) dosing a selected portion of tissue with a selected material; (5) implanting a medical device, (6) irradiating a selected portion of tissue with visible light, (7) irradiating a selected portion of tissue with IR light, and (8) irradiating a selected portion of tissue with radiation from a radioactive source.
